(19) United States
(12) Patent Application Publication
(10) Pub. No.: US 2014/0078278 A1
(43) Pub. Date: Mar. 20, 2014

(54) Systems and Methods for Controlling Lighting Strength of a Camera System by Time-Matched Intermittent Illumination

(71) Applicant: OmniVision Technologies, Inc., Santa Clara, CA (US)

(72) Inventor: Junzhao Lei, San Jose, CA (US)

(73) Assignee: OmniVision Technologies, Inc., Santa Clara, CA (US)

(21) Appl. No.: 14/019,137

(22) Filed: Sep. 5, 2013

Related U.S. Application Data
(63) Continuation-in-part of application No. 13/622,976, filed on Sep. 19, 2012.
(60) Provisional application No. 61/710,480, filed on Oct. 5, 2012.

(51) Int. Cl. A61B 1/06

(52) U.S. Cl. CPC A61B 1/0661
USPC 348/68

(57) ABSTRACT

A camera system with lighting strength control includes: an image sensor for capturing images of a scene; a light source for illumination of the scene; and a signal generator, in communication with the image sensor and the light source, for generation of (a) a first signal for controlling image capture by the image sensor and (b) a second signal for controlling a duty cycle of the light source. A method for controlling the lighting strength of a camera system, which includes an image sensor, an associated light source, and an associated signal generator, includes: (a) generating, using the signal generator, a first signal that controls image capture by the image sensor, and (b) generating, using the signal generator, a second signal that controls a duty cycle of the light source.

### CROSS-REFERENCE TO RELATED APPLICATIONS

The present application is a continuation in part of U.S. patent application Ser. No. 13/622,976 filed Sep. 19, 2012. The present application further claims the benefit of priority from U.S. Provisional Application No. 61/710,480 filed Oct. 5, 2012. Both of the above-identified applications are incorporated herein by reference in their entireties.

### BACKGROUND

Integrated imaging and lighting systems are used to record images in an otherwise dark environment. Common applications include medical endoscopes, snake inspection cameras, video borescopes, and machine vision. The lighting strength required to achieve a desired image brightness depends on a number of factors relating to the nature of the scene imaged, the configuration of the scene relative to both the imaging system and the lighting system, and the properties of the imaging and lighting systems. For instance, an object of a light color generally requires less bright illumination than an object of a darker color. Therefore, most systems include means for adjusting the lighting strength.

Medical endoscopes used to examine an interior part of the human body constitute an example where proper lighting strength is essential to reach the desired outcome, such as an accurate diagnosis or a successful operation. The operator of a medical endoscope regulates the power level of the light source to achieve the desired image brightness when moving the imaging system to examine different locations, or when targeting certain objects within a given scene.

### SUMMARY

In an embodiment, a camera system with lighting strength control includes: an image sensor for capturing images of a scene; a light source for illumination of the scene; and a signal generator, in communication with the image sensor and the light source, for generation of (a) a first signal for controlling image capture by the image sensor and (b) a second signal for controlling a duty cycle of the light source.

In an embodiment, a method for controlling the lighting strength of a camera system, which includes an image sensor, an associated light source, and an associated signal generator, includes: (a) generating, using the signal generator, a first signal that controls image capture by the image sensor, and (b) generating, using the signal generator, a second signal that controls a duty cycle of the light source.

### BRIEF DESCRIPTION OF THE DRAWINGS

FIG. 1 illustrates one exemplary endoscopy system including a camera module with an image sensor and a light source, according to an embodiment.

FIG. 2 illustrates one exemplary system for controlling lighting strength for images captured by an image sensor by time-matched intermittent illumination, according to an embodiment.

FIG. 3 illustrates exemplary cycles for signals used for controlling lighting strength for images captured by an image sensor by time-matched intermittent illumination, according to an embodiment.

FIG. 4 illustrates exemplary cycles for a signal for controlling a light source, all corresponding to the same duty cycle, according to an embodiment.

FIG. 5 illustrates one exemplary method for controlling lighting strength for images captured by an image sensor by time-matched intermittent illumination, according to an embodiment.

FIG. 6 illustrates one exemplary system for controlling lighting strength for images captured by an image sensor by time-matched intermittent illumination, according to an embodiment.

FIG. 7 illustrates one exemplary system for controlling lighting strength for images capturef by an image sensor by time-matched intermittent illumination, according to an embodiment.

FIG. 8 illustrates one exemplary system, including an image signal processor, settings, and a control panel, for controlling lighting strength for images captures by an image sensor by time-matched intermittent illumination, according to an embodiment.

FIG. 9 illustrates one exemplary method for starting up a system for controlling lighting strength for images captured by an image sensor by time-matched intermittent illumination, wherein settings are located in encrypted memory, according to an embodiment.

### DETAILED DESCRIPTION OF THE EMBODIMENTS

This invention relates to providing illumination for an image sensor operating in an otherwise dark environment. The illumination is provided by a light source that has two modes, on and off, and can operate at duty cycles between 0 and 100%. The lighting strength for frames captured by the image sensor is controlled by regulating the duty cycle of the light source. This is in contrast to conventional systems where the light source is on continuously and the power level is adjusted to provide a desired lighting strength. Duty cycle regulation requires fewer electronic components than power level adjustment as much of the functionality associated with duty cycle regulation may be performed by software/firmware. Further, duty cycle regulation is more efficient, in terms of power consumption, than conventional linear regulation schemes in which the light outputted by the light source is regulated by controlling power dissipation in a resistive devise. The present approach offers an efficient and very flexible solution that may be implemented with a minimum of electronic components. Importantly, consistent frame-to-frame lighting strength is easily achieved by matching the timing of the intermittent illumination to that of the image frame capture

The present invention has utility in camera systems situated in dark environments. Exemplary applications include, but are not limited to, endoscopes such as medical endoscopes, snake scope inspection systems, and borescopes, as well as non-scope inspection systems and surveillance systems.

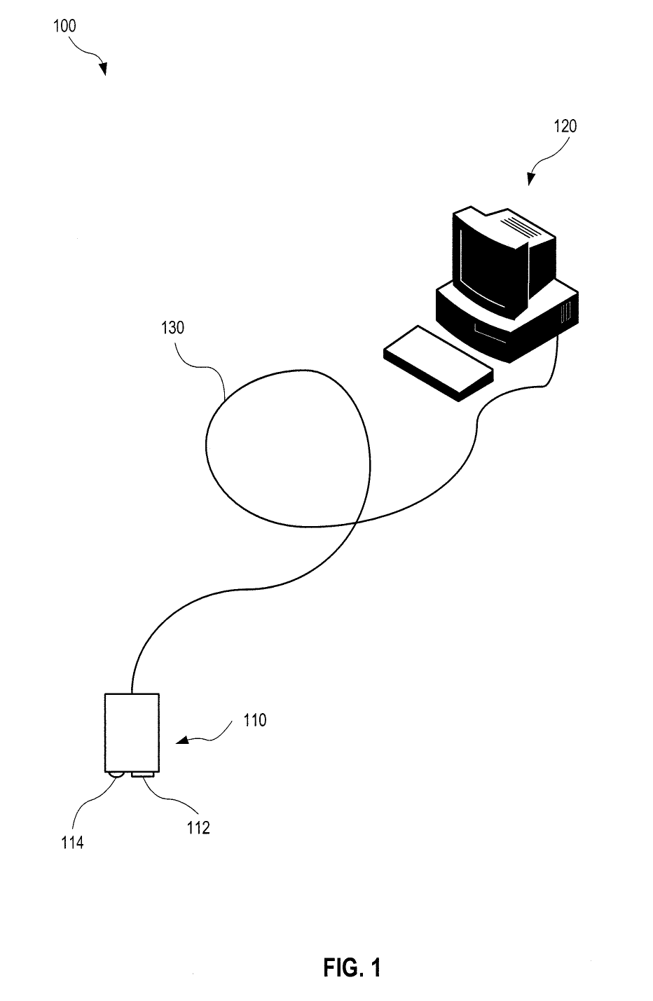

FIG. 1 illustrates an endoscopy system 100 including a camera module 110 according to the present invention. Camera module 110 is in communication with a control and display system 120 via a connector tube 130. Camera module 110 contains an integrated image and lighting system having an image sensor 112 and a light source 114. Light source 114 illuminates a scene imaged by image sensor 112. The present invention includes systems and methods that can be applied to controlling the lighting strength provided by light source 114 by intermittent illumination matched to the image capture rate of image sensor 112. In an embodiment, system 100 is a medical endoscope.

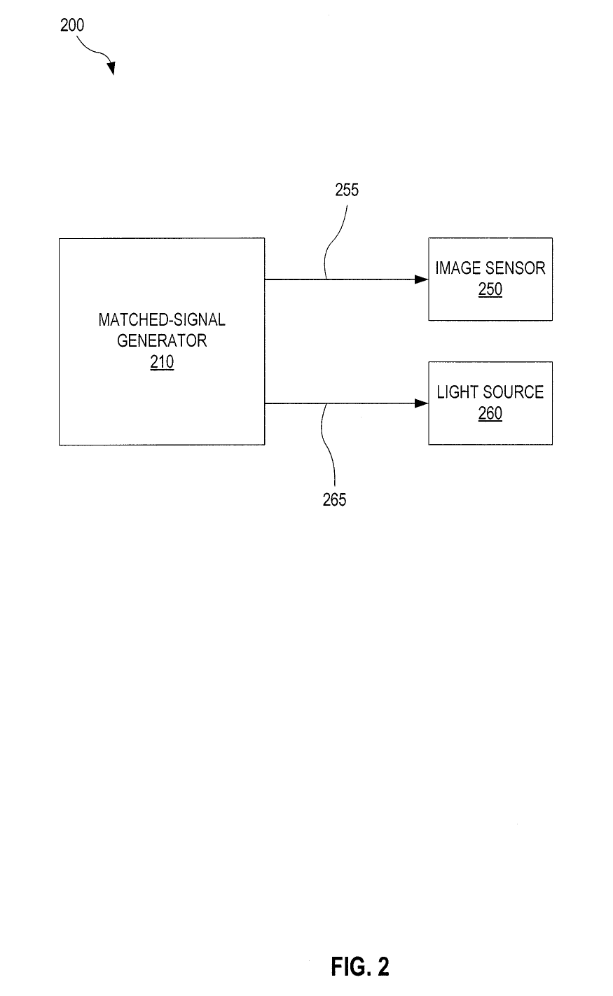

FIG. 2 shows a system 200 to control lighting strength for an imaging system by time-matched intermittent illumination. System 200 includes a matched-signal generator 210 in communication with an image sensor 250 and a light source 260. Image sensor 250 is, for example, a CMOS image sensor (CIS) or a CCD image sensor. In certain embodiments, light source 260 is a light emitting diode (LED). In certain other embodiments, light source 260 is an incandescent light source such as a halogen lamp. Matched-signal generator 210 outputs a trigger signal 255 to image sensor 250 for triggering of frame capture. Matched-signal generator 210 also supplies power to light source 260 in the form of a power signal 265. Power signal 265 can have two states: an “off” state that corresponds to the light source being off and an “on” state that corresponds to the light source being on at a preset strength.

In an embodiment, trigger signal 255 is periodic with a period T_{C}. This corresponds to image sensor 250 capturing images at a constant frame rate. Power signal 265 is periodic with the same period T_{C} as trigger signal 255. Power signal 265 is in its “on” state for an on-time Tₒₙ of light source 260, which can be expressed as

Tₒₙ=M×T_{FL}, Eq. 1

where M is a non-negative integer and T_{FL} is a fundamental light period that relates to the trigger signal period T_{C} through the equation

T_{C}=N×T_{FL}, Eq. 2

where N is a positive integer greater than or equal to M. The off-time for light source 260 is

T_{off}=(N−M)×T_{FL}, Eq. 3

and the duty cycle D for light source 260 is Equivalently, the lighting on-time

$$D = \frac{{T}_{on}}{{T}_{C}} = \frac{M}{N} . \qquad Eq . 4$$

frequency domain parameters:

$$\begin{matrix}{T}_{on} = M \times \frac{1}{{f}_{FL}} , & Eq . 5 a \\ {T}_{off} = ( N - M ) \times \frac{1}{{f}_{FL}} , & Eq . 5 b\end{matrix}$$

where f_{FL}=1/T_{FL} relates to the camera trigger frequency f_{C}=1/T_{C} through the equation

$${f}_{C} = \frac{{f}_{FL}}{N} . \qquad Eq . 6$$

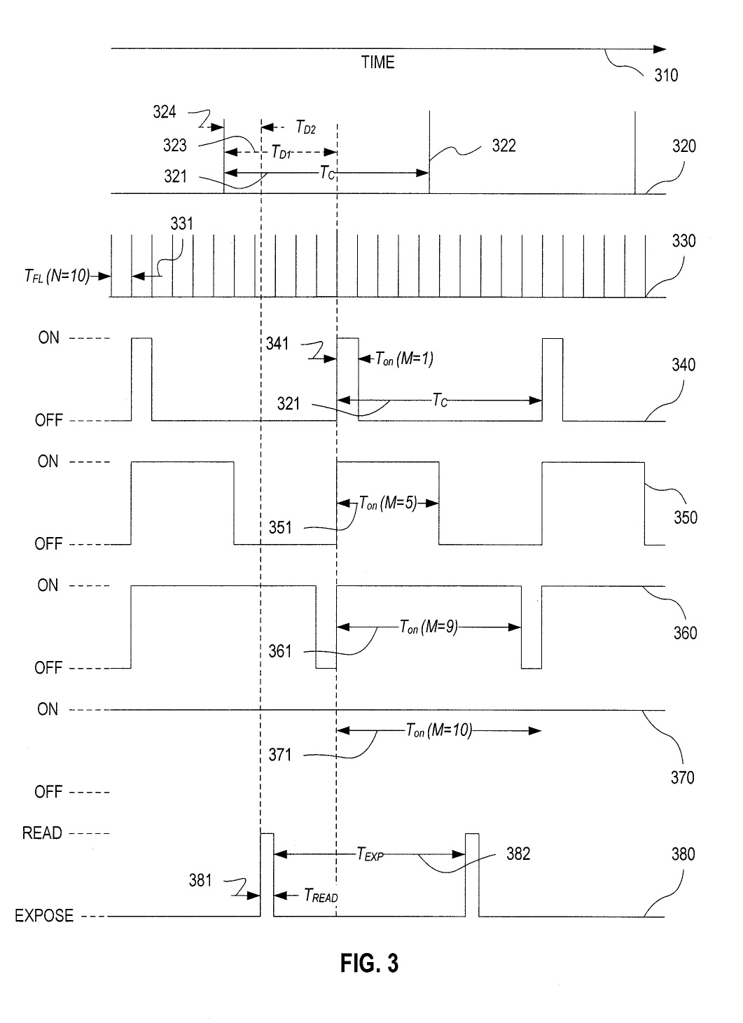

FIG. 3 illustrates non-limiting, exemplary cycles for trigger signal 255 and power signal 265 in accord with Eqs. 1 through 6. Standard image sensors consist of rows of pixels. The electrical charge accumulated by pixels during exposure is generally read out one row at a time. After readout of a given row, its pixels once again accumulate charge. In the following, the exemplary cycles displayed in FIG. 3 are first discussed in the context of a single row. Next, the discussion is extended to multiple rows in both a global and a rolling shutter regime.

Traces for all relevant signals are displayed as a function of time (310). Trace 320 shows the cycle for trigger signal 255 with a period T_{C} (321) between triggers (322). Trace 330 illustrates a periodic signal with fundamental light period T_{FL} derived from Eq. 2 for an exemplary value of N=10. In FIG. 3, the triggers 322 of trigger signal 255 (trace 320) and the waveform of the periodic signal illustrated as trace 330 are shown as delta functions. It is to be understood that either of these signals may have any appropriate waveform as known in the art, for instance a square wave, sawtooth, triangular, transistor-transistor logic (TTL), sinusoidal, or clock signal. Traces 340, 350, 360, and 370 show power signal 265 for exemplary values of M. Traces 340, 350, 360, and 370 are derived from the periodic signal illustrated as trace 330, as prescribed by Eq. 1 for M=1, M=5, M=9, and M=10, respectively. The lighting strength progressively increases from trace 340 through trace 370 as the duty cycle for light source 260 increases from 10% (trace 340), to 50% (trace 350), to 90% (trace 360), and to 100% (trace 370). An optional delay T_{D1} 323 represents the delay between a trigger (322) of trigger signal 255 (trace 320) and the onset of the on-state of power signal 265 (traces 340, 350, 360, and 370). Trigger signal 255 (trace 320) triggers readout of pixels, as illustrated with trace 380, with an optional delay T_{D2} between trigger event 322 and the start of a readout period T_{READ} (381). Between readout periods 381, the pixels are exposure in an exposure period T_{EXP} (382).

While FIG. 3 illustrates cycles for the specific value of N=10, the discussion is readily extended to other N values. Greater values of N provide higher resolution for lighting strength regulation.

The requirement of identical periodicities of trigger signal 255 and power signal 265 ensures consistent frame-to-frame lighting strength. If this requirement was not fulfilled, the overlap between the on-time Tₒₙ of light source 260 and capture of individual frames by image sensor 250 would vary from frame to frame, leading to varying frame-to-frame lighting strength as the two unmatched periodicities shift in and out of phase with each other. The identical periodicities of the present invention maintain a constant phase overlap between the on-time Tₒₙ of light source 260 and frame capture by image sensor 250.

Note that the onset of the on-state of power signal 265 need not coincide with a trigger event of trigger signal 255. This is illustrated in FIG. 3 where the onset of the on-state for all of traces 340, 350, 360, and 370 are shifted by a delay T_{D1} (323) relative to a trigger event of trigger signal 255 (trace 320). Consistent frame-to-frame illumination is maintained for any value of T_{D1}. Similarly, the actual image exposure (382) may be offset in time from the corresponding trigger 322 by a delay T_{D2} (324) with no effect on the consistency of frame-to-frame lighting strength.

The above discussion of FIG. 3 is applicable without modification to exposure and readout of multiple rows in the global shutter regime, in which all rows are sequentially read out followed by simultaneous exposure of all rows. In this case, trace 380 is representative of every row with T_{READ} being the total readout time for all rows.

Rolling shutter image sensors apply a rolling readout and exposure process wherein, concurrently with the readout of one row, all other rows are exposed. When readout of one given row is completed, it returns to exposure while the next row is read out, etc. This eliminates the overhead associated with global shutters, in which all but one row are idling while the one row is read out. As a result, higher sensitivity for a given frame rate can be achieved using a rolling shutter. Most commonly used image sensors, particularly in the more affordable price range, are configured with a rolling shutter. Since individual rows are not synchronously exposed in a rolling shutter sensor, different rows may potentially be associated with different lighting conditions. In situations where the exposure time is much greater than the readout time, this effect is negligible.

A significant benefit of the present invention is that the intermittent nature of the light source enables use of an image sensor configured with a rolling shutter while achieving consistent row-to-row lighting strength. In an embodiment, the image sensor, e.g., image sensor 250 (FIG. 2) is configured with a rolling shutter and delays T_{D1} and T_{D2} are controlled to avoid overlap between image readout and the on-time of the light source. When this condition is met, the function of a rolling shutter is equivalent to that of a global shutter.

Traces 340 and 350 of FIG. 3 are examples of light source cycles that provide consistent row-to-row lighting strength in an embodiment with a rolling shutter image sensor, since there is no overlap between image readout (381) and the on-time of the light source. In the cases of traces 360 and 370 (FIG. 3), on the other hand, the light source is on during image readout. Therefore, when using a rolling shutter, different rows may be exposed under different lighting conditions, resulting in inconsistent row-to-row lighting strength.

Since the timing of light source on-time and image capture are based on a common clock signal, e.g., trigger signal 255 (trace 320 of FIG. 3), the present invention inherently facilitates control of delays without the need for added features such as additional electronic circuitry. This simplifies the control of relative delays between light source control signals and image capture, especially for use scenarios requiring a relatively low frame rate. Such use scenarios include, but are not limited to, applications where the image output is a video stream the rate needed for the video stream to appear smooth to a human observer defines the frame rate requirement. Exemplary applications include endoscopes such as medical endoscopes. A minimum of 24 frames per second is required to produce a smooth video. Medical endoscopes frequently operate with a frame rate of 30 frames per second. In an embodiment, image sensor 250 is configured with a rolling shutter and operates at frame rates in the range from 24 to 200 frames per second. In another embodiment, image sensor 250 is configured with a rolling shutter and operates at frame rates in the range 24 to 1000 frames per second.

In another embodiment, a global shutter image sensor is used, e.g., image sensor 250 is configured with a global shutter. In this case, consistent lighting strength for all rows is an inherent consequence of the system design. Global shutter image sensors may be advantageously used at high frame rates.

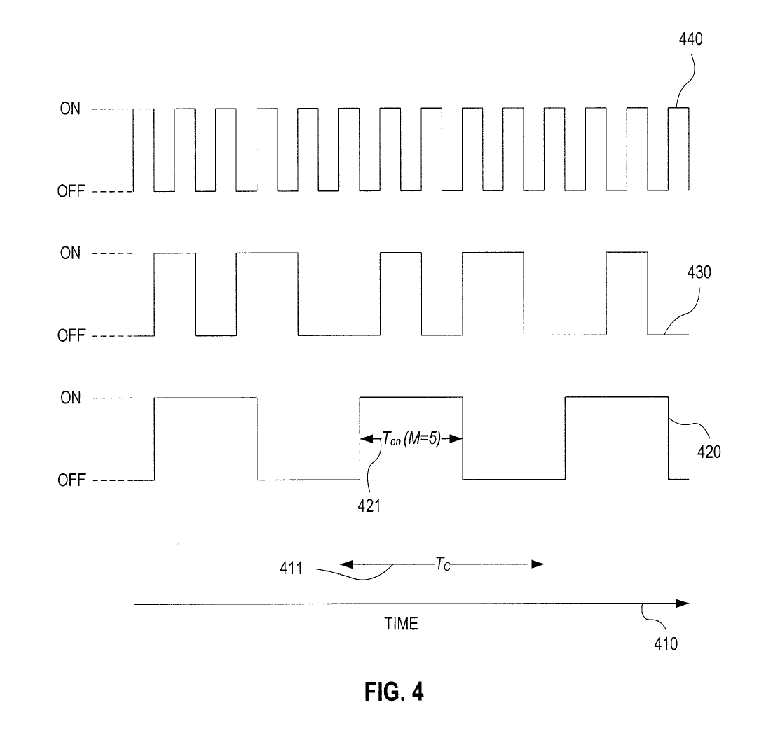

Any given on-time Tₒₙ may be achieved either as a single, contiguous on-time as shown in FIG. 3 or as the sum of several shorter on-times. FIG. 4 illustrates non-limiting examples hereof for N=10 and M=5, i.e., a 50% duty cycle. Three cycles, each resulting in a 50% duty cycle, are illustrated as traces 420, 430, and 440 as a function of time (410) for a trigger signal period T_{C} (411). Each trace is generated from the same period T_{FL}, e.g., the periodic signal illustrated as 330 of FIG. 3. Trace 420 utilizes a single on-pulse (421) to achieve the 50% duty cycle. In trace 430, two pulses of differing durations together yield a 50% duty cycle. In trace 440, a periodic pulse train forms the 50% duty cycle.

The embodiment expressed by Eqs. 1 through 6 is advantageous as it is easily implemented in a system consisting of only few electronic components, while providing flexibility by allowing for different values of M and N that can be changed through either hardware, software, or a combination thereof.

While FIGS. 2, 3 and 4 are discussed in the context of a power signal 265 controlling the on- and off-times for light source 260, it is to be understood that other methods may be applied to turn on and off light source 260 without departing from the scope of the present invention. Such methods include, but are not limited to, a physical shutter controlled electronically or a strobe wheel. However, these mechanical methods have limitations and/or disadvantages compared to strictly electronic control. For instance, both shutters and strobe wheels constitute an additional mechanical element that occupies space and are associated with wear and tear that may be substantial for a video capture application. Strobe wheels are configured for operation at a set duty cycle, or a series of preconfigured duty cycles, which limits the flexibility of the system. Further, a change of duty cycle requires a mechanical operation on the strobe wheel or a strobe wheel replacement.

In an embodiment, trigger signal 255 is not periodic. However, Eqs. 1 through 4 still hold true with T_{C} interpreted as an exposure time, or an exposure and readout time, for a frame captured by image sensor 250. This embodiment applies to a use scenario in which the frame rate of image sensor 250 is not constant. Images may be captured at varying frame rates and/or on demand, e.g., when prompted by an operator or an external trigger event. Referring to the illustration in FIG. 3, when operating in this mode, consistent frame-to-frame lighting strength relies on the delays T_{D1} (323) and T_{D2} (324), and the on-time Tₒₙ being such that the on-time Tₒₙ associated with a given image exposure does not overlap with other image exposures.

Matched-signal generator 210 of FIG. 2 may include software, firmware, a computer, and other electronic circuitry. An operator may control aspects of matched-signal generator 210. For example, the operator may change the duty cycle of light source 260, in accordance with Eqs. 1 through 3, to attain a certain image brightness. In another example, certain aspects of the functionality of matched-signal generator 210 are preset and, e.g., configured in electronic circuitry within matched-signal generator 210. Optionally, matched-signal generator 210 includes auto brightness control. In one embodiment, the auto brightness control is based on analysis, performed by matched-signal generator 210, of images captured by image sensor 250 and subsequent adjustment of the duty cycle of light source 260 as prescribed by Eqs. 1 through 3. In another embodiment, the auto brightness control utilizes a separate element, such as a photodiode, to provide a brightness measure to matched-signal generator 210, which then adjusts the duty cycle of light source 260 accordingly.

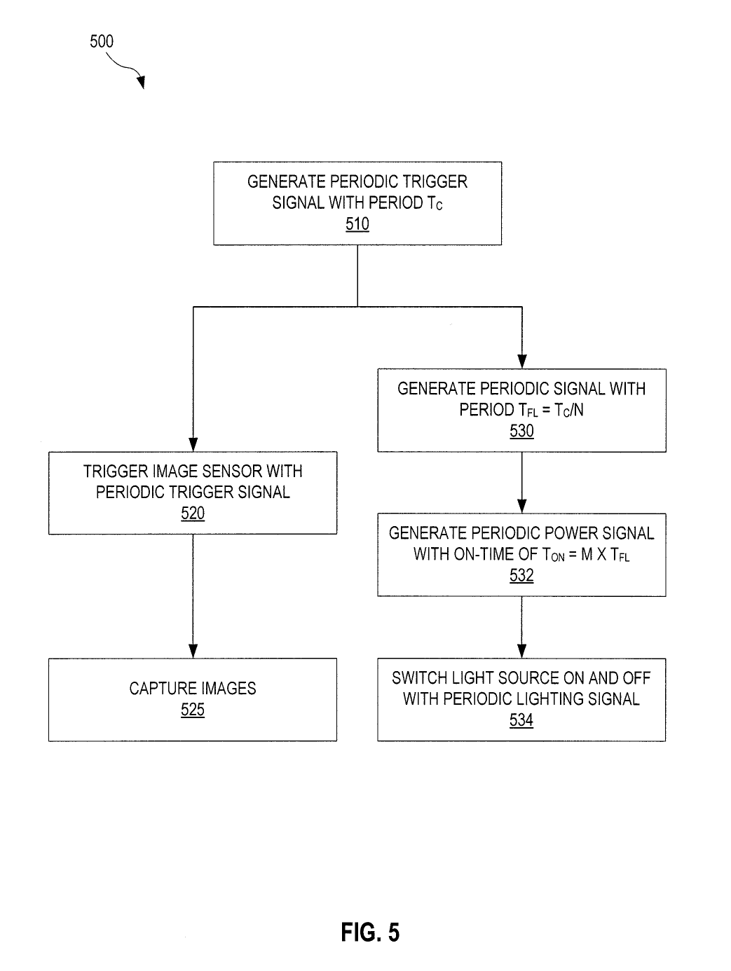

FIG. 5 illustrates a method 500 for controlling the lighting strength for an imaging system according to Eqs. 1 through 6. In a step 500, a periodic trigger signal, e.g., trigger signal 255 (FIG. 2), with period T_{C} is generated. This signal is used in two portions of method 500 that may be performed in parallel: steps 520 and 525 for controlling image capture and steps 530, 532, and 534 for controlling associated lighting. In step 520, the periodic trigger signal triggers the image sensor (e.g., image sensor 250). In step 525, images are captured at the rate defined by the periodic trigger signal. In step 530, the trigger signal generated in step 510 is used to generate a periodic signal with a period T_{FL}, e.g., a fundamental light period that relates to T_{C} as expressed in Eq. 2. In step 532, the periodic signal generated in step 530 is used to generate a periodic power signal (e.g., lighting signal 265 of FIG. 2), which has an on-time Tₒₙ as prescribed by Eq. 1. In a step 534, the periodic lighting signal switches on and off the light source, e.g., light source 260.

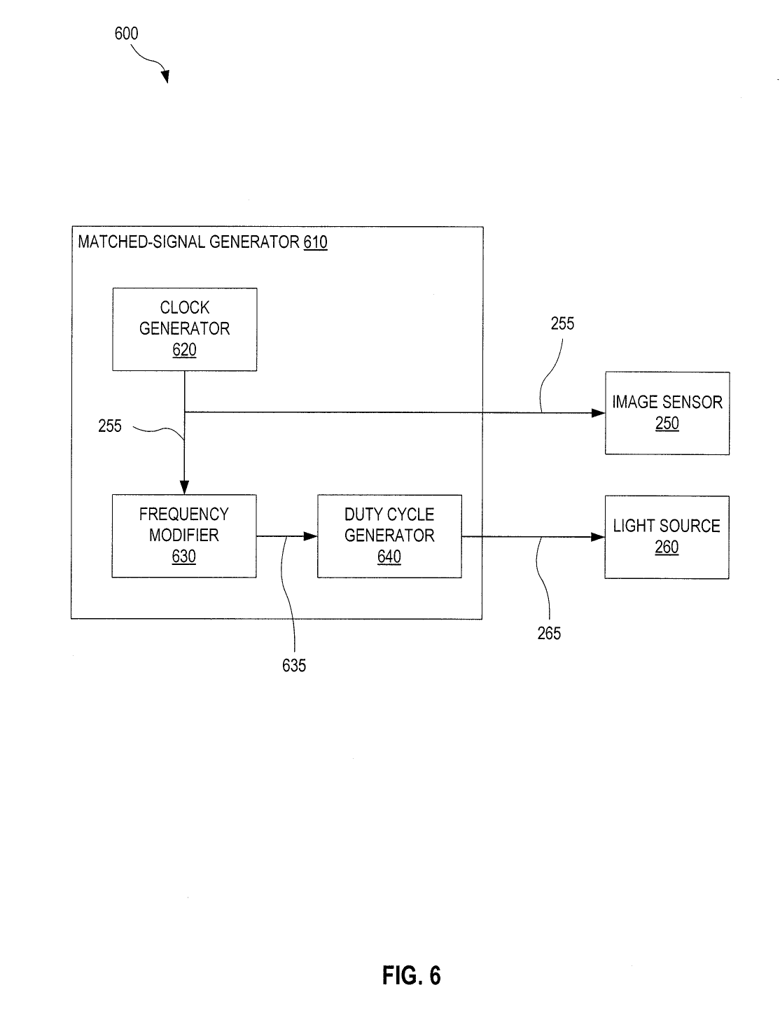

FIG. 6 shows a system 600 that is an embodiment of system 200 of FIG. 2 and utilizes, for example, method 500 of FIG. 5. In system 600, a matched-signal generator 610 includes a clock generator 620 that outputs trigger signal 255, i.e., clock signal generator 620 performs step 510 of method 500. Matched-signal generator 610 is an embodiment of matched-signal generator 210 of FIG. 2. In system 600, trigger signal 255 is periodic with a period T_{C}. Trigger signal 255 is relayed to image sensor 250, as discussed for FIG. 2, to perform performs steps 520 and 525 of method 500. Trigger signal 255 is also communicated to a frequency modifier 630 that rate-multiplies trigger signal 255 to output a fundamental light signal 635. The period of fundamental light signal 635, T_{FL}, relates to T_{C} as expressed by Eq. 2. Equivalently, the frequency of fundamental light signal 635, f_{FL}, relates to the camera trigger frequency f_{C} as expressed by Eq. 5. Accordingly, frequency modifier 630 performs step 530 of method 500. Fundamental light signal 635 is sent to a duty cycle generator 640 that outputs power signal 265 based thereupon and in accordance with Eq. 1. That is, duty cycle generator 640 performs step 532 of method 500. Duty cycle generator 640 sends power signal 265 to light source 260 to perform step 534 of method 500.

In an embodiment, frequency modifier 630 is a standard rate multiplier or frequency divider as known to a person skilled in the art. Likewise, clock generator 620 may be a standard clock generator module as known in the art.

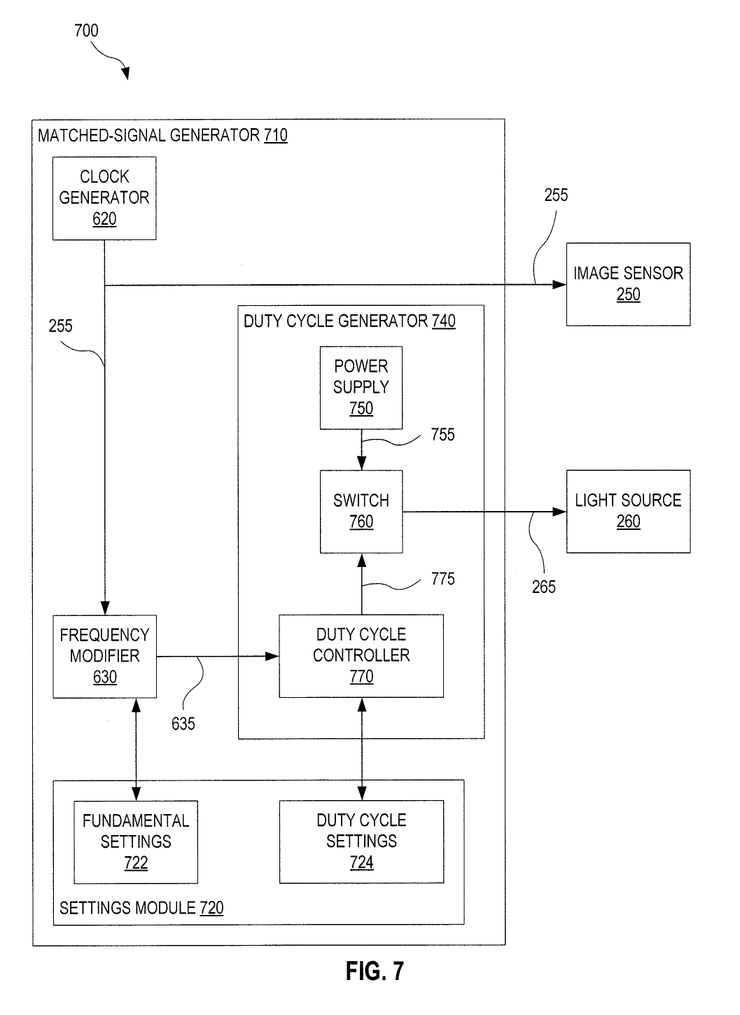

FIG. 7 illustrates an embodiment of system 600 of FIG. 6 as a system 700. System 700 includes a matched-signal generator 710 that is an embodiment of matched-signal generator 610 of FIG. 6. Matched-signal generator 710 includes a duty cycle generator 740 constituting an embodiment of duty cycle generator 640 of FIG. 6. Duty cycle generator 740 includes a power supply 750 connected to light source 260 through a switch 760. When switch 760 is closed, power supply 750 provides power 755 for light source 260. A duty cycle controller 770 generates a switching signal 775 based partly on fundamental light signal 635. Switching signal 775 controls switch 760 to relay power 755 supplied by power supply 750 to light source 260 as power signal 265. In an embodiment, duty cycle controller 770 is a computer, a microprocessor, a central processing unit (CPU), or a combination thereof. In certain embodiments, duty cycle controller 770 includes a user interface such that users can control at least portions of the functionality of duty cycle controller 770.

A settings module 720 includes fundamental settings 722 and duty cycle settings 724. In one embodiment, settings module 720, or portions thereof, is integrated in the system providing duty cycle controller 770. Fundamental settings 722 are accessible by frequency modifier 630 and include a value for the positive integer N of Eq. 2 to generate the desired harmonic of trigger signal 255 according to Eq. 2. Similarly, duty cycle settings 724 are accessible by duty cycle controller 770 and include a value for the non-negative integer M of Eq. 1 to generate the desired duty cycle according to Eqs. 1 and 3. In certain embodiments, fundamental settings and duty cycle settings, or portions thereof, are configurable by an operator. In an example, an operator may choose from a library of settings, i.e., values for N and M obeying Eq. 6, to achieve a certain lighting strength.

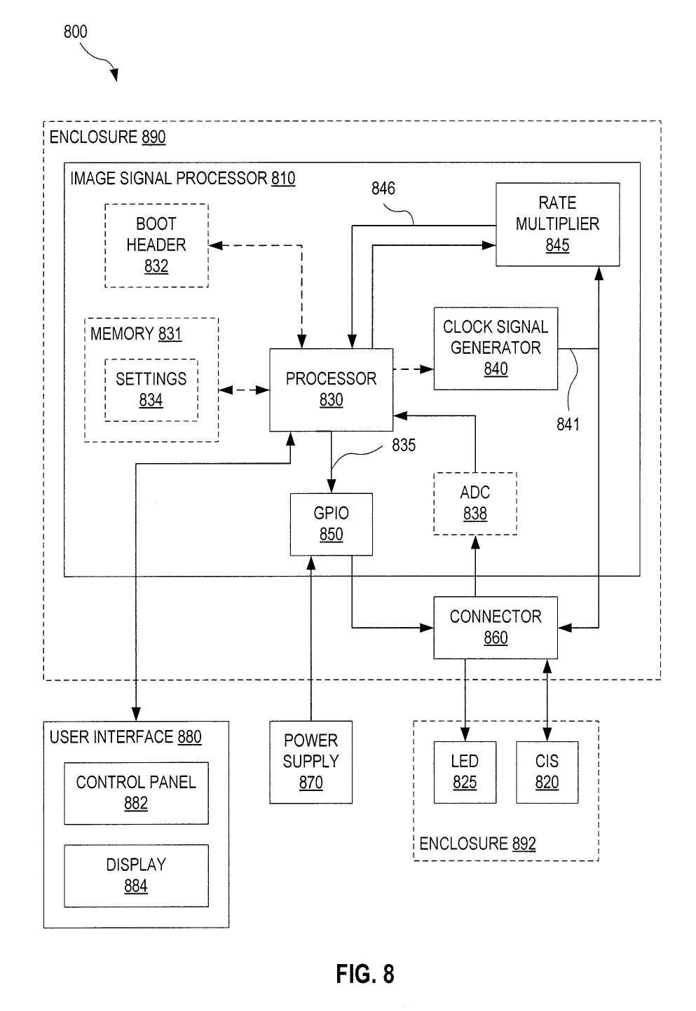

FIG. 8 shows a system 800 for controlling lighting strength for an image sensor by time-matched intermittent illumination, in accord with systems 200, 600, and 700 of FIGS. 2, 6, and 7, respectively and in accord with method 500 of FIG. 5. System 800 includes an image signal processor (ISP) 810 in communication with a CIS 820 and an LED 825 via connector 860. CIS 820 and LED 825 are embodiments of image sensor 250 and light source 260, respectively, of FIG. 2. In an embodiment, ISP 810 is part number OV570 from OmniVision Technologies. LED 825 provides lighting of a scene imaged by CIS 820. ISP 810 is further in communication with a power supply 870 and a user interface 880. User interface 880 includes a control panel enabling a user to change the lighting strength provided by LED 825, and a display 884 for displaying images, e.g., video, captured by CIS 820.

A clock signal generator 840 and a rate multiplier 845 included in ISP 810 are capable of generating the time-matched timing signals required for accomplishing a desired lighting strength through intermittent illumination by LED 825. Clock signal generator 840 outputs a periodic clock signal 841 that is communicated via connector 860 to CIS 820 and to rate multiplier 845. Periodic clock signal 841 is, e.g., trigger signal 255 of FIGS. 2, 6, and 7 with a period T_{C}. In an embodiment, the period T_{C} is a preset property of clock signal generator 840. In another embodiment, period T_{C} is communicated to clock signal generator 840 by a processor 830 included in ISP 810. Rate multiplier 845 communicates to processor 830 a periodic light signal 846, e.g., fundamental light signal 635 of FIGS. 6 and 7, that is a harmonic of periodic clock signal 841 as expressed in Eq. 2. The order of the harmonic, e.g., the value of N in Eq. 2, is communicated to rate multiplier 845 by processor 830. Processor 830 processes periodic light signal 846 received from rate multiplier 845 to generate a switching signal 835, e.g., switching signal 775 of FIG. 7. In certain embodiments, switching signal 835 and periodic light signal 846 correspond to Tₒₙ and T_{FL}of Eq. 1 and relate to each other through the value of M as expressed in Eq. 1.

Switching signal 835 functions as a control input for a general purpose input/output port (GPIO) 850. GPIO 850 is connected to a power supply 870 and, via connector 860, to LED 825. In this configuration, GPIO 850 is operated as a switch such that switching signal 835 controls when power flows from power supply 870 to LED 825. A GPIO is a special type of port because it is capable of floating an output without causing error. For example, for a GPIO 850 it is permissible to either be connected or not connected to LED 825. This provides flexibility to the system by allowing LED 825 to be disconnected. In one embodiment, GPIO 850 is a transistor gate.

Processor 830 is in communication with a user interface 880 that includes a control panel 882 and a display 884. Processor 830 is further in communication with an optional boot header 832 and/or an optional memory 831 that includes an optional settings module 834. Settings required for processor 830 to control the generation of periodic signal 841 and switching signal 835 are provided to processor 830 from control panel 882, optional settings module 834, or a combination thereof. In certain embodiments, settings module 834 contains a collection of settings for generating periodic signal 841 and switching signal 835, e.g., T_{C}, N, and M. These settings are communicated to control panel 882 via processor 830, where an operator may select specific settings that are subsequently communicated back to processor 830.

Optional memory 831 may be part of ISP 810, as shown in FIG. 8, or be located externally to ISP 810. In certain embodiments, optional memory 831 is a detachable electronic non-volatile computer storage device, for instance of the type erasable programmable read-only memory (EPROM), flash memory, non-flash electrically erasable programmable read-only memory (EEPROM), programmable read-only memory (PROM), field programmable read-only memory (FPROM), or one-time programmable non-volatile memory (OTP NVM). In one embodiment, optional memory 831 is an EPROM device that is in communication with processor 830 via an I²C interface. Information is transferred from the EPROM device to processor 830 one byte at a time. In another embodiment, optional memory 831 is a flash memory device that is in communication with processor 830 via an SPI interface. In this case, information is transferred to processor 830 in blocks of 512 bytes. This results in a faster transfer than that achieved with the EPROM device. However, the EPROM device offers a more affordable solution.

Images recorded by CIS 820 are relayed to processor 830 via connector 860. In one embodiment, CIS 820 outputs image information in analog format, which is then converted by an optional analog to digital converter (ADC) 838 to digital format readable by processor 830. In this embodiment, CIS 820 and ADC 838 may be, respectively, part number OV6930 and part number OV420, both from OmniVision Technologies. In other embodiments, CIS 820 includes ADC circuitry, in which case optional ADC 838 is omitted. Finally, processor 830 relays digital images to display 884.

ISP 810 and connector 860 are contained by an optional enclosure 890, together forming a control box for CIS 820 and LED 825. CIS 820 and LED 825 are contained in another optional enclosure 892. In particular embodiments, enclosure 892, with CIS 820 and LED 825, is the integrated imaging and lighting system of a medical endoscope. User interface 880 may be located externally to optional enclosure 890, for example on a separate computer, portable digital assistance (PDA), tablet computer, or a smart phone and optionally utilizing a processor thereof. User interface 880 communicates with processor 830 using any one of methods known in the art including, but not limited to, wired interfaces such as USB, Ethernet, FireWire, MIDI, or Thunderbolt, and wireless protocols such as Wi-Fi, Bluetooth, or radio-frequency. Alternatively, user interface 880 is integrated within enclosure 890 and, optionally, utilizing processor 830 for all its processing needs. Power supply 870 may be located within optional enclosure 890 or externally thereto.

In certain embodiments, for instance as applied in capsule endoscopes, ISP 810, connector 860, power supply 870, CIS 820, and LED 825 are integrated into a single enclosure. In this embodiment, processor 830 may be in wireless communication with control panel 882 and/or display 884; or settings may be preloaded onto ISP 810 as part of settings module 834 and/or recorded images stored within memory 831.

In another embodiment, memory 831 includes algorithms (not shown in FIG. 8) for automatically adjusting the lighting strength by choosing appropriate settings from settings module 834 as conditions change. In yet another embodiment, such algorithms are located externally to ISP 810, for example as part of control panel 882.

System 800 facilitates encryption of optional memory 831 in order to prevent duplication of, e.g., settings 834 as well as prevent use of unauthorized and/or counterfeit product in the place of the intended version of optional memory 831. Standard encryption protocols as known by a person skilled in the art may be employed. In one embodiment, boot header 832, which is accessible only by processor 810, includes address information for an encryption key located on memory 831. Only a valid encryption key will allow operation of ISP 810.

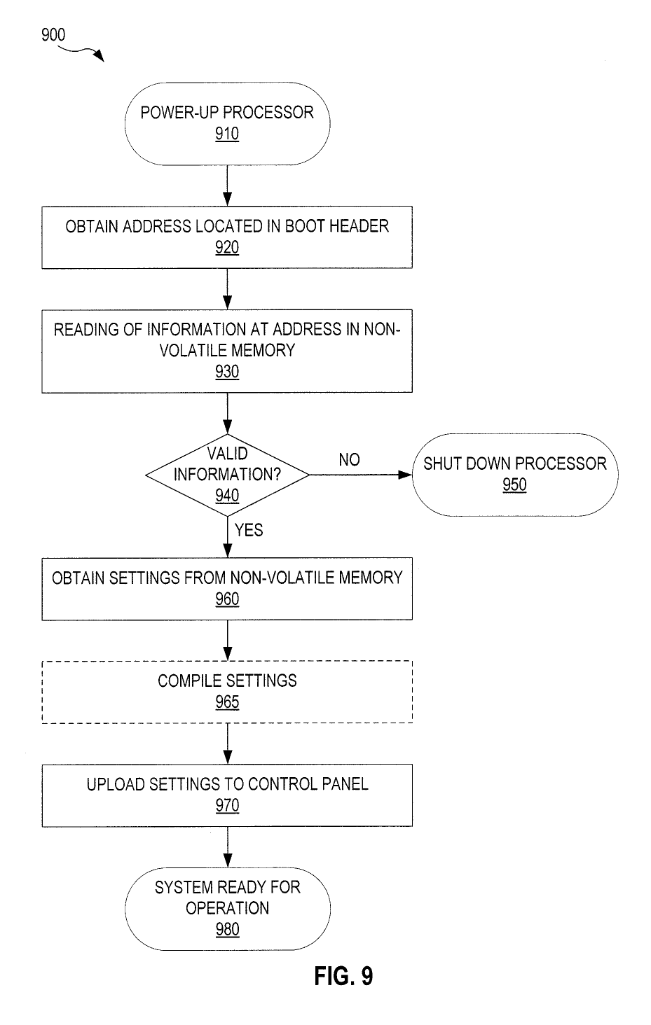

FIG. 9 illustrates an exemplary start-up procedure, method 900, for a system utilizing encrypted memory. In a step 910, the processor of the system, e.g., processor 830 of system 800 (FIG. 8), is powered up. In a step 920, the processor obtains an address from its associated boot header, e.g., boot header 832 (FIG. 8). In a step 930, the processor reads the information on associated non-volatile memory, e.g., memory 831 of FIG. 8, located at the address obtained in step 920. In a step 940, the information obtained in step 930 is evaluated by the processor (e.g., processor 830) and compared to information in its boot header (e.g., boot header 832). If the information is not a valid code, the processor is shut down in a step 950. If the information is indeed a valid code, the processor obtains settings located in the non-volatile memory, e.g., settings 834 of memory 831 (FIG. 8). In an optional step 965, the processor compiles the settings. In an embodiment, the settings are stored in the non-volatile memory in assembly language and compiled by the processor to a language readable by the control panel. In a step 970, the settings are uploaded to a control panel, e.g., control panel 882 (FIG. 8), where after the system is ready for operation in a step 980.

Changes may be made in the above methods and systems without departing from the scope hereof. It should thus be noted that the matter contained in the above description and shown in the accompanying drawings should be interpreted as illustrative and not in a limiting sense. The following claims are intended to cover generic and specific features described herein, as well as all statements of the scope of the present method and system, which, as a matter of language, might be said to fall therebetween.

Combinations of Features

Features described above as well as those claimed below may be combined in various ways without departing from the scope hereof. For example, it will be appreciated that aspects of one system or method for controlling lighting strength described herein may incorporate or swap features of another system or method for controlling lighting strength described herein. The following examples illustrate possible, non-limiting combinations of embodiments described above. It should be clear that many other changes and modifications may be made to the methods and system herein without departing from the spirit and scope of this invention:

(A) A camera system with lighting strength control may include an image sensor for capturing images of a scene and a light source for illumination of the scene.

(B) In the system denoted as (A), the image sensor may capture a single image for each period of a duty cycle of the light source.

(C) The system denoted as (A) may further include a signal generator, in communication with the image sensor and the light source, for generation of a first signal for controlling image capture by the image sensor and a second signal for controlling a duty cycle of the light source.

(D) In the camera system denoted as (C), the first and second signals may be periodic and share a common period.

(E) In the system denoted as (D), the image sensor may capture a single image for each common period.

(F) In the systems denoted as (C), (D), and (E), on and off states of the light source may correspond to a first and a second state, respectively, of the second signal.

(G) In the system denoted as (F), the total duration of the first state of the second signal, during one common period, may be one unit fraction or multiple unit fractions of the common period.

(H) In the systems denoted as (C) through (G), the signal generator may include a clock signal generator for generating the first signal.

(I) In the systems denoted as (C) through (H), the signal generator may include a frequency modifier.

(J) In the system denoted as (I), the frequency modifier may be in communication with the clock signal generator for generating a multiplied signal that is a harmonic of the first signal.

(K) The system denoted as (J) may include a duty cycle generator, in communication with the frequency multiplier, for processing of the multiplied signal to generate the second signal.

(L) In the systems denoted as (A) through (K), the image sensor may have a rolling shutter.

(M) In the systems denoted as (A) through (K), the image sensor may have a global shutter.

(N) In the systems denoted as (A) through (M), the light source may be adapted to be in an off-state during image readout.

(O) The systems denoted as (A) through (N) may be implemented in a medical endoscope.

(P) The systems denoted as (A) through (O) may include non-volatile memory capable of storing encrypted duty-cycle settings for the light source.

(Q) The system denoted as (P) may include a processor capable of decoding the encrypted duty-cycle settings.

(R) The system denoted as (Q) may include a control panel for choosing a specific one of the decoded, encrypted duty cycle settings.

(S) The system denoted as (P) may include a control panel for choosing a specific one of the encrypted duty cycle settings.

(T) A method for controlling the lighting strength of a camera system, which includes an image sensor, an associated light source, and an associated signal generator, may include generating, using the signal generator, a first signal controlling image capture by the image sensor.

(U) The method denoted as (T) may include generating, using the signal generator, a second signal controlling a duty cycle of the light source.

(V) In the methods denoted as (T) and (U), the first signal may be periodic with a first signal period.

(W) In the method denoted as (U), the first signal may be periodic with a first signal period, and the second signal may be periodic with the first signal period

(X) In the methods denoted as (V) and (W), the total duration of an on-state of the second signal, within a first signal period, may be one unit fraction or multiple unit fractions of the first period.

(Y) The methods denoted as (W) and (X) may include generating a multiplied signal having a period that is a unit fraction of the first signal period

(Z) In the method denoted as (Y),the second signal may be generated such that each period of the multiplied signal corresponds to either an on-state or an off-state of the second signal.

(AA) The methods denoted as (X) through (Z) may include providing duty cycle settings corresponding to combinations of settings for (a) the first setting period, (b) the value of the unit fraction, and (c) the number of unit fractions during which the second signal is in an on-state

(AB) The method denoted as (AA) may include selecting a specific one of the duty cycle settings.

(AC) In the methods denoted as (AA) and (AB), providing duty cycle settings may include decoding encrypted data.

(AD) The methods denoted as (T) through (AC) may include capturing images, using the image sensor, of a scene illuminated by the light source.

(AE) In the methods denoted as (V) through (AD), a single image may be captured for each first period.

(AF) In the methods denoted as (T) through (AE), the image sensor may be configured with a rolling shutter.

(AG) In the methods denoted as (T) through (AE), the image sensor may be configured with a global shutter.

(AH) In the methods denoted as (U) and (W) through (AG), the second signal may be in an off-state during readout of images captured by the image sensor.

(AI) In the methods denoted as (U) and (W) through (AG), the light source may be off during readout of images captured by the image sensor.

(AJ) The methods denoted as (T) through (AI) may be implemented in a medical endoscope.

What is claimed is:

1. A camera system with lighting strength control, the camera system comprising:
an image sensor for capturing images of a scene;
a light source for illumination of the scene; and
a signal generator, in communication with the image sensor and the light source, for generation of (a) a first signal for controlling image capture by the image sensor and (b) a second signal for controlling a duty cycle of the light source.

2. The system of claim 1, the first and second signals being periodic and sharing a common period.

3. The system of claim 2, the image sensor capturing a single image for each common period.

4. The system of claim 3, wherein the on and off states of the light source correspond to a first and a second state, respectively, of the second signal, the total duration of the first state of the second signal, during one common period, being one unit fraction or multiple unit fractions of the common period.

5. The system of claim 4, the signal generator comprising:
a clock signal generator for generating the first signal;
a frequency modifier, in communication with the clock signal generator, for generating a multiplied signal that is a harmonic of the first signal; and
a duty cycle generator, in communication with the frequency multiplier, for processing of the multiplied signal to generate the second signal.

6. The system of claim 1 being implemented in a medical endoscope.

7. The system of claim 4, further comprising:
non-volatile memory capable of storing encrypted duty-cycle settings;
a processor capable of decoding the encrypted duty-cycle settings; and
a control panel for choosing a specific one of the decoded, encrypted duty cycle settings.

8. A method for controlling the lighting strength of a camera system comprising an image sensor, an associated light source, and an associated signal generator, the method comprising:
generating, using the signal generator, a first signal controlling image capture by the image sensor; and
generating, using the signal generator, a second signal controlling a duty cycle of the light source.

9. The method of claim 8, the first signal being periodic with a first signal period.

10. The method of claim 9, wherein the second signal is periodic with the first signal period, and the total duration of an on-state of the second signal, within a first signal period, is one unit fraction or multiple unit fractions of the first signal period.

11. The method of claim 10, further comprising generating a multiplied signal having a period that is a unit fraction of the first signal period, and wherein the second signal is generated such that each period of the multiplied signal corresponds to either an on-state or an off-state of the second signal.

12. The method of claim 11, further comprising capturing images, using the image sensor, of a scene illuminated by the associated light source.

13. The method of claim 12, wherein a single image is captured for each first period.

14. The method of claim 10, further comprising:
providing duty cycle settings corresponding to combinations of settings for (a) the first signal period, (b) the value of the unit fraction, and (c) the number of unit fractions during which the second signal is in an on-state; and
selecting a specific one of the duty cycle settings.

15. The method of claim 14, wherein providing duty cycle settings comprises decoding encrypted data.

16. The method of claim 10 being implemented in a medical endoscope.
